(19) United States
(12) Patent Application Publication
(10) Pub. No.: US 2022/0118001 A1
(43) Pub. Date: Apr. 21, 2022

(54) METHODS AND USES OF NATURAL BASED COMPOSITIONS

(71) Applicant: ASSOCIATION FOR THE ADVANCEMENT OF TISSUE ENGINEERING AND CELL BASED TECHNOLOGIES & THERAPIES A 4TEC, Barco Guimaraes, Braga (PT)

(72) Inventors: Simone DOS SANTOS SILVA, Braga (PT); Luísa Cidália GUIMARÄES RORIGUES, Fafe (PT); Emanuel MOUTA FERNANDES, Vila Verde (PT); Rui Luís CONÇALVES DOS REIS, Porto (PT)

(21) Appl. No.: 17/432,840

(22) PCT Filed: Feb. 20, 2020
(86) PCT No.: PCT/IB2020/051442

§ 371 Date: Aug. 20, 2021

(30) Foreign Application Priority Data
Feb. 20, 2019 (PT) 115323
Dec. 31, 2019 (EP) 19220278.6

(51) Int. Cl. A61K 31/722; A61K 36/886; A61K 36/889; A61K 47/38; A61K 9/70; A61K 9/06; A61K 47/10

(52) U.S. Cl. CPC A61K 31/722; A61K 36/886; A61K 36/889; A61K 9/7023; A61K 9/7007; A61K 9/06; A61K 47/10; A61K 47/38

(57) ABSTRACT

The present disclosure relates to a composition for use in tissue engineering and/or regenerative medicine comprising a compound selected from chitosan, aloe vera, ethylcellulose, cellulose derivatives, or mixtures thereof; and coconut oil or virgin coconut oil. Films, or membranes, and oleogels comprising the aforementioned composition are also disclosed. Furthermore, the methods to prepare these structures as well as their use in tissue engineering are also encompassed. The products of the invention were developed to overcome the use of surfactants due to its harmful effects, especially when regarding biological applications. The compositions are then useful in tissue engineering solutions.

### TECHNICAL FIELD

The present disclosure relates to obtaining 2D or 3D structures, namely films, membranes, and oleogels from natural compositions in a result of the combination of virgin coconut oil with biomacromolecules, and the methods to obtain it. The proposed formulations could contain or not additives to facilitate the processing, promote better performance properties, or induce any additional characteristics.

The developed compositions in the form of 2D or 3D structures are useful for tissue engineering applications and/or regenerative medicine.

### BACKGROUND ART

Emulsions are a class of colloids in which the stable coexistence of two or more immiscible phases occurs. However, as it is well known in the art, typically emulsions are unstable; hence, emulsifying agents must be added to form a stable emulsion (1). When a surface-active substance is added to, for example, a mixture of oil and water, a monolayer of surfactants is spontaneously formed with their hydrophilic ends facing the water phase and their hydrophobic ends submerged in the oil phase. The formation of the surfactant monolayer decreases the surface tension at the oil-and-water interface and determines the kinetic stability of the system. However, the presence of those surfactants may be considered not beneficial specially when is considered biological applications. These compounds may become systemic substances along with the beneficial agents offsetting his benefits by the harmful effects these surfactants can cause.

Moreover, a further approach is described in the literature (2), which relates to the formation of a stable mixture of natural origin polymer with vegetable oil, without an emulsifying agent. Such a stable mixture occurs only at a restricted polymer/oil ratio range between 0.5-1, above which phase separation became evident during drying. The interaction between some natural polymers and oils may be due to the polycationic nature of the polymer and the oppositely charged lipids responsible for the electrostatic interaction between the polymeric chains and the oil, which leads to its stability.

Virgin coconut oil (VCO), a vegetable oil, is composed of almost 90-95% saturated fatty acids, such as lauric acid (LAC, 45-53%), caprylic acid, and capric acid. It also contains large amounts of triglycerides, proteins, antioxidants, and vitamin E. The metabolic and physiological properties of LAC account for many of the features of coconut oil. Coconut oil is rapidly metabolized because it is easily absorbed, and LAC is easily transported. VCO can be extracted by any coconut source by direct pressing technique. At low temperature, VCO is solid or nearly solid, slow to oxidize and has a long shelf-life.

The health benefits of VCO such as anti-fungal, antibacterial, anti-inflammatory, anti-aging, and anti-oxidant properties have established its role in the cosmetic, pharmaceutical and food industry; however, VCO is mainly used in the food industry. Despite its use as cooking oil, nutritional supplement, personal care ingredient, and antimicrobial agent, VCO has also been used in health-promoting, disease prevention, and medication. The European market for VCO has grown significantly over the last years, mainly due to increasing consumer attention to healthier diets. Beyond its applications in the food and cosmetics industries, VCO has been studied for other uses, mainly in the biomedical area.

Studies on animal models confirmed the anti-inflammatory, analgesic, and antipyretic properties of VCO. In acute inflammatory models, VCO displayed moderate anti-inflammatory effects on ethyl phenylpropionate-induced ear edema in rats, and carrageenan- and arachidonic acid-induced paw edema. VCO also showed a moderate analgesic effect on the acetic acid-induced writhing response as well as an antipyretic effect in yeast-induced hyperthermia.

Health and healing properties of coconut oil are studied in its pure state, i.e. in liquid form, or mixed with other ingredients such as oil materials, film formers, surfactants, or other adjunct components. US 2010/0021563 A1 from Jan. 28, 2010 describes compositions comprising coconut oil, as the active agent and at least one additional active component, as zinc salt, wherein the compositions include emulsions, creams, lotions, and gels intended for topical application for treatment and/or prevention of diseases. In another document, US 2016/0206548 A1 from Jul. 21, 2016 were described compositions where coconut oil composition is incorporated into a substrate to form a wet wipe for delivery to the skin.

Natural compositions comprising VCO combined with biomacromolecules, namely chitosan, ethylcellulose, or mixtures thereof, may impart natural, medicinal, pharmaceutical, and methods for use. In this sense, those combinations will provide ways to develop medical devices through modulation of these intrinsic properties, ensuring successful application in the tissue engineering field.

Chitosan (CHT) is composed of randomly distributed 3-(1-4)-linked D-glucosamine (deacetylated unit) and N-acetyl-D-glucosamine (acetylated unit). CHT is derived from chitin that is obtained from shells of crustaceans such as crabs, shrimp, and squids. Such CHT powder is commercially available with different deacetylation degree (from 75 to 95%) and different molecular weights. CHT is also available in a medical-grade, for example, under the trade name chitoceuticals with a deacetylation degree from 70 to 95%.

CHT is a polysaccharide, water-insoluble but soluble in weak organic acid solutions. CHT has excellent properties, namely film-forming ability, biocompatibility, biodegradability, and anti-bacterial properties. It has been proposed as a biomaterial and valuable alternative to the existing commercial materials. The versatility of CHT structure, characteristics, and properties had allowed the development of different architectures for tissue engineering of bone, cartilage, and skin. Dressing materials based on CHT, for instance, are well-known on the market, mainly in Japan and the USA.

Recently, the oil encapsulation or combinations of oils with CHT has been proposed, opening new ways to process it. However, these studies are centered on their use in food and cosmetic formulations. Production of CHT based films enriched with essential oils such as anise, orange, cinnamon, and lemon essential oils improve the natural antimicrobial properties of chitosan for food coating.

Addition of plant extracts as aloe vera (AV), a tropical plant, into the formulations can bring more advantages to the emulsion systems due to its intrinsic properties such as antibacterial action, anti-tumor, immunostimulatory, anti-bacterial, wound healing, antioxidant, and anti-inflammatory activity, which associated with the emulsion system promoted the required properties for preventing and treating skin diseases, as, e.g., psoriasis and diabetic foot ulcers.

AV belongs to the lily (Liliaceae) family. It is often recognized to be the most versatile known medicinal plant in nature. AV contains a diverse composition, including anthraquinones, vitamins, minerals, enzymes, amino acids, and polysaccharides. AV leaf is composed of two main parts, the outer green rind and the colourless inner parenchyma that contain a clear viscous gel. The AV gel is composed mainly of water (about 97-99%) and several phytochemical substances.

AV is widely used in products such as tonics, tablets, capsules, and supplements. It is also found as an essential component in hair tonics and masks, shampoos, and sunblocks. Some of the AV related documents on the mentioned uses (WO 2008097109 A1, CA 2915328 A1) are linked to chemical composition and intrinsic features. Besides its applications in the food and cosmetics industries, its beneficial effects have been studied on wound infections, burn wounds, diabetes, and cancer. The topical application of AV is known as a natural remedy. AV can infiltrate into the skin tissue and act on the wound healing process as a whole.

In material science and engineering, AV-based matrices can be produced alone or through its combination with recognized polymers from synthetic and/or natural origin. For example, blended membranes composed by AV gel and CHT developed by the solvent casting technique have better physical (roughness, degradation rate, wettability, mechanical properties) and cellular response than CHT alone. These membranes could be used as active wound dressing materials. These findings prospect an advantageous effect from the incorporation of AV into the emulsion films, envisioning its biomedical application.

Edible oleogels have also been prepared by combining ethylcellulose (EC) with an edible oil with or without surfactants. EC is a derivative of cellulose in which some of the hydroxyl groups on the repeating glucose units are converted into ethyl ether groups, of which the number can vary. Also, ethylcellulose is a GRAS (generally regarded as safe) material for use in food products.

EC undergoes a thermoreversible sol-gel transition in the presence of liquid oil. This unique behavior is based on the polymer's ability to associate through physical bonds. Recently, EC-based oleogels have been used as a replacement for fats in foods, as heat-resistance agents in chocolate, as oil-binding agents in bakery products, and as the basis for cosmetic pastes.

WO 2010/143066 A1 from 16 Dec. 2010 provides an edible oleogel consisting of one or more oils or fats, ethylcellulose, and a surfactant, wherein heating the EC/oil/surfactant mixture to a temperature above the glass transition temperature of the ethylcellulose with mixing, followed by cooling to form an oleogel. The resulting oleogel is homogeneous, elastic, substantially anhydrous, and has a gelation temperature below 100° C. It can be used as a fat substitute in foods. The useful concentration range is between 4% and 20% (w/w) EC in oil, while EC 10 cP, 22 cP, and EC 45 cP are desirable polymers to be used in food products.

EC oleogels are preferently applied for food and cosmetic industries. The incorporation of VCO into the formulation may enlarge the range of application of the achieved materials from food to the biomedical field. The EC/VCO oleogels synergistically denotes the EC and VCO properties, earlier described such as antimicrobial action, moisturizing, anti-inflammatory activity.

Additives or adhesion promoters can also be added to natural compositions for improving the miscibility, adhesion, and flexibility of their constituents. Additives such as glycerol, glycerine, bio-based plasticizers, and green plasticizers act as adhesion promoters for VCO and biomacromolecules such as AV, CHT, and EC. Glycerol, a kind of small polyalcohol molecule, can be used as a plasticizer to improve mechanical properties of films with non-toxic effects on the human body. The additives such as glycerol can be added directly into the formulations in different quantities or ratios between the components. Glycerol has been used as an emulsifying agent, texturizer, and plasticizer to modify the texture of foods and emulsions. Glycerol can alter the magnitude of the repulsive and attractive forces governing the stability and rheological properties of an emulsion.

These facts are disclosed to illustrate the technical problem addressed by the present disclosure.

### General Description

The present disclosure relates to obtaining 2D or 3D structures, namely films, membranes, and oleogels from natural compositions in a result of the combination of virgin coconut oil with biomacromolecules, and the methods to obtain it. More specifically, the compositions present the mixture of at least two constituents, being one of them virgin coconut oil. The components are mixed in different conditions and further processed using solvent casting, freeze-drying, compression moulding, salt leaching, or in combination, to obtain materials with different shapes, sizes, and properties. The proposed formulations could contain or not additives to facilitate the processing, promote better performance properties, or induce any additional characteristics.

In an embodiment, the key aspects of the present disclosure are:
Chitosan/Virgin Coconut Oil (VCO) emulsion films are characterized as superabsorbent materials. This feature can be modulated by the addition of glycerol or derivatives to emulsions;
Ethylcellulose/virgin coconut oil oleogels with different textures can be produced using gelation or compression moulding process;
Compression moulding combined with salt leaching process may be used to produce ethylcellulose/virgin coconut oil porous oleogels;
The presence of additives as glycerol in the preparation of the formulations influenced: i) positively the interaction between the constituents of the emulsion system; ii) increases the flexibility of the films; iii) increases the hydrophilicity of the oleogels; and iv) modulates the mechanical properties of the oleogels;
The stiffness of the new oleogels can be tuned by (a) use of ethylcellulose with different molecular weight; (b) decreasing the cooling temperature from 6° C. up to −20° C.; (c) use plasticizer agent such as glycerol and (d) addition salt particles in different sizes to create porosity into the oleogel structure.

The developed compositions in the form of 2D or 3D structures are useful for tissue engineering applications.

The present disclosure is related to the use of natural-based compositions combining biomacromolecules, with coconut oil (CO), preferably virgin coconut oil (VCO) in 2D or 3D structures, namely films, membranes and oleogels, for tissue engineering applications.

In the present disclosure, the term additives mean small molecules or crystalline structures that are applied to improve the properties and textures of the developed materials.

The term surfactant means compounds that lower the surface tension (or interfacial tension) between two liquids, between a gas and a liquid, or between a liquid and a solid.

The term plasticizer means additives that increase the plasticity, decrease the viscosity, or increase the hydrophilicity of the materials.

The term pore salt means small crystalline particles that will be added into the formulation process to create porosity.

In an embodiment, the inventive step of this work is related not only to their composition and methods but also to their potential biomedical use. With this intent, compositions for producing 2D structures, in particular non-porous and porous films, were composed of at least two components, with or without the presence of additives.

In another embodiment, the composition can include natural polymers combined with various vegetable oils and additives that form emulsion systems with or without the presence of additives.

In another embodiment, the structures will be produced using any of the standard techniques based on solvent casting, emulsion process, compression moulding and freeze-drying.

In another embodiment, compositions for producing 3D-based structures, namely non- and porous oleogels should have at least two constituents, with or without the presence of additives. The oleogels are composed, preferably by ethylcellulose (EC) or cellulose derivatives and VCO or CO, using a gelation process. EC/VCO and EC/CO oleogels can be alternatively produced applying a melt-based process by compression molding to the gelation system. In that process, parameters such as pressure, temperature and time would modulate the properties of the produced oleogels.

In an embodiment, the application of the compression molding process in the production of EC/VCO oleogels would bring advantages; namely, high production volume, good flexibility in part design, shrinkage in the product is reduced, and minimum raw material waste.

Additional additives may be used to tune a certain level of porosity in an oleogel. In a salt leaching procedure, the salt particles having a suitable particle size are added to the composition of the material, wherein the substrate formed using, per example solvent casting or compression moulding, may be immersed in water to promote the leaching out of the salt particles, creating a porous structure.

In another embodiment, the methodology of compression moulding together with salt leaching leads to porous induction into the 3D-architectures, which makes them useful for tissue engineering applications as it allows cell culture and cell penetration. Similar behaviour is expected when solvent casting and salt leaching methodologies are combined.

In an embodiment is disclosed a method to obtain 2D or 3D structures, namely films or oleogels, which combine at least two components, comprising the steps of:
(i) mixing the components;
(ii) solvent casting;
(iii) compression moulding or gelation;
(iv) optionally salt leaching;
or any combination of the steps above.

In an embodiment, the present disclosure relates to a composition for use in tissue engineering comprising a compound selected from the following list: chitosan, aloe vera, ethylcellulose, cellulose derivatives, or mixtures thereof; and coconut oil, preferably virgin coconut oil in a concentration ranging from 1 to 5% (v/v).

In another embodiment, the composition further comprises an additive, wherein the additive does not comprise surfactants. The additive is selected from a list consisting of salt, glycerol, glycerine, or mixtures thereof. The composition comprises at least 1% (w/w) of additive, preferably 1-65% (w/w).

The present disclosure also relates to films, membranes or oleogels comprising the composition described in the previous embodiments.

In an embodiment, the films or membranes comprise at least one of the following combinations:
chitosan and virgin coconut oil;
chitosan and coconut oil;
chitosan, aloe vera, and virgin coconut oil;
chitosan, aloe vera, and coconut oil.

In an embodiment, the film or membrane comprises 94-99% (v/v) of chitosan, preferably 96-99% (v/v), more preferably 97-99% (v/v); and 1-5% (v/v) of virgin coconut oil or coconut oil, preferably 1-3% (v/v), more preferably 1-2% (v/v). The film or membrane may further comprise at least 1% (v/v) of a glycerol-based plasticizer.

In a yet another embodiment, the film or membrane further comprises 63-65% (v/v) of chitosan, preferably 64-65% (v/v); 31-33% of aloe vera, preferably 31.5-33% (v/v), more preferably 32-33% (v/v); 1% (v/v) of plasticizer-based on glycerol; and 1-5% (v/v) of virgin coconut oil or coconut oil, preferably 1-2% (v/v).

In an embodiment, the volume ratio between the chitosan and aloe vera is 1.5:1-2.5:1, preferably 2:1.

In another embodiment is disclosed an oleogel structure comprising: ethylcellulose and virgin coconut oil, or ethylcellulose and coconut oil. A preferred embodiment relates to oleogels comprising 15-30% (w/w) of ethylcellulose, cellulose derivatives, or mixtures thereof, preferably 15-20% (w/w); and 70-85% (w/w) of virgin coconut oil or coconut oil, preferably 80-85% (w/w); and an additional volume of a plasticizer comprising glycerol or its derivates.

An aspect of the present disclosure comprises the method to prepare films or membranes and oleogels, the method comprising: (i) mixing two or more components selected from the following list: virgin coconut oil, coconut oil, chitosan, aloe vera, ethylcellulose, cellulose derivatives, glycerol, glycerine, salt or mixtures thereof, wherein at least one of the components is coconut oil, in particular, virgin coconut oil; (ii) homogenizing the mixture; (iii) solvent casting or freeze-drying; and (iv) optionally compression molding or gelation, preferably in the presence of additives.

In a further embodiment is disclosed a method comprising the step of adding salt particles of suitable size, in order to create porosity. The salt particles are further removed by salt leaching, a step of immersion in distilled water for several hours or several days.

In an embodiment, the structures are dried at room temperature, in particular, wherein the room temperature is 25° C.

In a further embodiment is disclosed a method wherein the cooling temperature varies between −20° C. and 25° C.

In a further embodiment is disclosed a method, in which the freeze-drying method uses temperatures in a range between −20° C. e−196° C.

In an embodiment, the VCO concentration varies between 1 and 85% (v/v), preferably 1%-5% (v/v).

In a further embodiment, it is also disclosed a structure obtainable from the process described in any of the embodiments of the present subject matter. The structure is porous and/or non-porous; and is a film, or membrane or an oleogel;

In an embodiment, the composition related to the present disclosure is for tissue engineering applications.

The present disclosure also comprises a patch, a kit or a scaffold comprising the composition, film or oleogel related to the present subject matter.

In a further embodiment, is disclosed the use of the structures obtained from the disclosed process in tissue engineering applications.

A further embodiment relates to the use of the structure obtained as described in the previous embodiments, as a support for cell culture and/or cell differentiation used with different cell lines and primary cells, such as human fibroblasts and/or mesenchymal stem cells, preferably mesenchymal stem cells derived from adipose tissue.

### BRIEF DESCRIPTION OF THE DRAWINGS

The following figures provide preferred embodiments for illustrating the disclosure and should not be seen as limiting the scope of the invention.

Scheme 1 is a schematic representation of an embodiment of the processes involved in the production of the 2D and 3D structures.

FIG. 1 illustrates an embodiment of an emulsion system composed by chitosan and virgin coconut oil (a) and viscosity curves as a function of shear rate of the individual components and the emulsion systems with different percentages of VCO (b).

FIG. 2 illustrates an embodiment of the emulsion films: (a) chitosan, (b) chitosan/virgin coconut oil, (c) chitosan/virgin coconut oil with the addition of glycerol, and (d) chitosan/aloe vera/virgin coconut oil; and (e,f) a representative image of the flexibility of the emulsion films.

FIG. 3 illustrates an embodiment of emulsion films: alginate/VCO (a), silk fibroin/VCO (b), and gelatin/VCO (c).

FIG. 4 illustrates an embodiment of the morphology of the emulsion films surface: chitosan (a), chitosan/VCO (1% v/v) (b), and chitosan/VCO (5% v/v) (c).

FIG. 5 illustrates an embodiment of swelling behaviour of the emulsion films prepared without glycerol (a), with the addition of glycerol (b) before and after 30 minutes in water, swelling (%) data on emulsion films (c) and the respective expansion factor (d).

FIG. 6 illustrates an embodiment of chitosan/VCO based emulsion films cytotoxicity. (a) Cell viability on extracts of the chitosan/VCO based emulsion films assessed by MTS assay during 72 hours of culture (b) Cell damage assessed by F-actin staining (cytoskeleton, light grey) and counterstained with DAPI staining (nuclei, white) during 72 hours of culture (scale bar: 50 μm).

FIG. 7 illustrates an embodiment of oleogels (3D structure) prepared using ethylcellulose/VCO according to different methodologies and conditions: room temperature (a), 4° C. (b), compression moulding (c) and compression moulding with salt leaching (d).

FIG. 8 illustrates an embodiment of morphological features of the porous-based oleogels produced by compression moulding and salt leaching. Scale bar: 250 μm.

FIG. 9 illustrates an embodiment of oleogels (3D structure) prepared using ethylcellulose/coconut oil (a) and ethylcellulose/almond oil (b). Both prepared by compression moulding with salt leaching in the presence of glycerol.

FIG. 10 illustrates an embodiment of mechanical properties of oleogels prepared by compression moulding using different temperatures and with or without glycerol.

FIG. 11 illustrates an embodiment of human adipose stem cells (hASCs) viability on ethylcellulose/VCO oleogels. Cell damage assessed on oleogels by F-actin staining (cytoskeleton, light grey) and counterstained with DAPI staining (nuclei, white): (a) ethylcellulose/VCO 15/85 and (b) ethylcellulose/VCO 20/80; (c) Cell viability of hASCs on extracts of ethylcellulose assessed by MTS assay up to 72 hours of culture and (d) Cell viability of hASCs on direct contact ethylcellulose/VCO based oleogels assessed by MTS assay up to 7 days of culture. Scale bar: 50 μm.

### DETAILED DESCRIPTION

The present disclosure relates to a composition for use in tissue engineering comprising a compound selected from chitosan, aloe vera, ethylcellulose, cellulose derivatives, or mixtures thereof; and coconut oil or virgin coconut oil. Films or membranes and oleogels structures based on the aforementioned composition are also disclosed.

Furthermore, the methods to prepare these structures as well as their use in therapy are also encompassed. The products of the invention were developed to overcome the use of surfactants due to its harmful effects, especially when regarding biological applications. The compositions are then useful in tissue engineering solutions. Films, oleogels, patchs, kits or scaffolds comprising the composition of the present disclosure are also considered.

It is also disclosed the use of the structures related to the present subject matter as support for cell culture and/or cell differentiation used with different cell lines and primary cells, such as human fibroblasts and/or mesenchymal stem cells, preferably mesenchymal stem cells derived from adipose tissue.

In an embodiment, the materials used were the following: chitosan from crab shells (practical grade; Sigma Aldrich) with a degree of deacetylation of 85% was used after purification by a re-precipitation method; virgin coconut oil (VCO, Copra Indústria Alimentícia Ltda, Brasil); coconut oil (CO, Ktc, Brasil); ethylcellulose (Sigma Aldrich), viscosity grade (molecular weight between 4 cP to 100 cP); salt particles (NaCl, 355-1000 μm); glycerol (Sigma Aldrich), reagent grade and used as received.

Emulsion films and oleogels structures were produced using the methodologies A and C as schematically shown on Scheme 1.

In one embodiment, there is the production of emulsion films. CHT solutions with a 1% (w/v) concentration were prepared in acetic acid solution (1% v/v). The solution was left to homogenize overnight at room temperature. Further, the solution was filtered through a sintered glass filter to remove undissolved impurities. Different quantities of VCO (1-5% v/v) were separately added to CHT solution and homogenized at 15500 rpm for 10 min using a high-speed homogenizer (Ultra Turrax, T18 Basic, IKA-Werke GmbH & Co. KG, Germany), to obtain a stable emulsion solution. Additional components namely AV gel or glycerol may be added into the system.

Further, the emulsion systems are spread into small petri dishes and dried at room temperature or 37° C. (solvent casting). Alternatively, emulsion solutions may be spread into small petri dishes and submitted to freeze-drying to obtain porous emulsion films. Freeze-drying is also a useful technique to obtain an aerogel. This technique is a drying method largely applied to reduce water activity and the susceptibility of the materials to bacterial attack. It is based on freezing the polymeric solution at temperatures varying between −20° C. and −196° C. to allow the growth of ice crystals, followed by the removal of the solvent, which is generally water, through lyophilization. Different variables affect the morphology of the structures obtained. Fast-freezing, for example, could allow better preservation of the fine structure of the gels.

In one embodiment, there is the production of 3D-based structures. EC/VCO oleogels may be made by a process in which EC, VCO and optional additional constituents or additives such as glycerol are mixed at a speed between 100 and 700 rpm at a temperature above the softening point of the EC polymer (120-190° C.), until its dissolution in the oil. Then, the solution is transferred for a stainless (spherical, cylindrical, cubic, or rectangular) mold. Further, the molds may be cooled at several temperatures in the range of −20° C. up to 25° C.

For compression molding, a similar procedure was used, wherein EC and VCO were mixed using a speed between 500 and 600 rpm at 170-190° C., during 1-2 h. Glycerol may be added into the mixture, followed by closing the mold during 30-60 minutes under 130° C. The sample is then cooled while inside of the mold until reaching the room temperature. Further, the material can be cooled using a range of 25° C. up to −20° C. to obtain different properties.

In one embodiment, porous oleogels were produced using a salt leaching technique. Salt particles (45-90% w/w) were mixed with EC/VCO system. Further, the compression molding process may be replaced by a gelation process and spread it on a dry plate. After curing, the resulting material can be immersed in distilled water for several hours or days to remove the salt particles. After salt removal, the oleogels may be dried at room temperature.

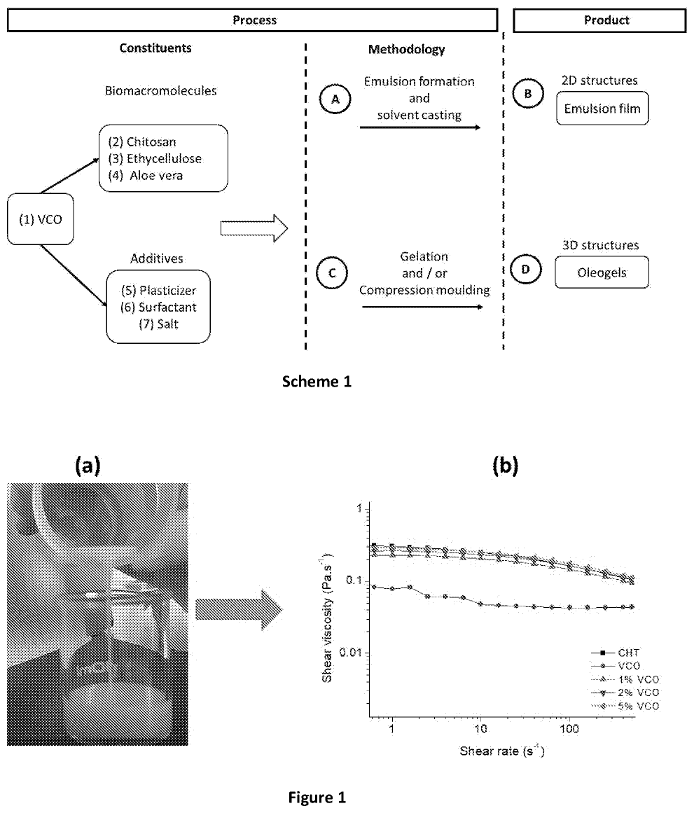

The following description concerns the characterization of the solutions used to obtain 2D or 3D structures, namely films or membranes, and oleogels. Rheological measurements were conducted on a Malvern Instruments Kinexus rheometer equipped with a stainless steel parallel-plate sample holder of 20 mm in diameter and a 1 mm gap. Steady-state flow measurements were carried out at 25° C. in the range of 1-100 s⁻¹ during 5 min, and rheological parameters (shear stress, shear rate, viscosity) were obtained from the software rSpace. All characterizations were performed 3 times for each sample. The achieved results are illustrated in FIG. 1b.

In an embodiment, the analysis of the viscosities as a function of the shear rate of the independent constituents and the emulsion systems solutions revealed a non-Newtonian behavior (FIG. 1b). The viscosities of the emulsion systems solutions are at the same range of chitosan alone, with some small differences between them with the increase in the VCO concentration.

In an embodiment, the process used for producing emulsion films based on VCO and CHT was solvent casting. In the process of solvent casting, it is possible to obtain the film product directly after the evaporation of the solvent on its final shape. Membranes can be produced using solvent casting with or without porogens, freeze-drying technique, spin casting, and electrospinning. Solvent casting is the most commonly used technique for the preparation of polymeric membranes, owing to the advantage of producing large and constant surface areas. This method consists of polymer dissolution in an appropriate solvent and subsequent solvent evaporation. Additionally, drugs or specific molecules can be incorporated during the dissolution of the polymer or even on the casted film. The membrane structure and its properties are influenced by many experimental factors such as choice of solvent and non-solvent, polymeric solution composition, and humidity. Furthermore, instead of using a unique polymer, two or more polymers can be mixed to form blends which are useful in the production of membranes with desirable properties. Many polymeric membranes have been produced using polysaccharides, proteins, or their combinations envisioning biomedical tissue engineering applications.

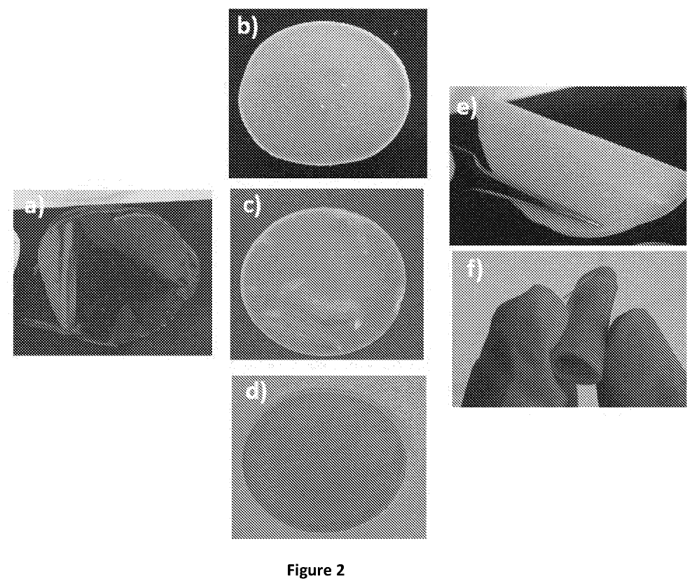

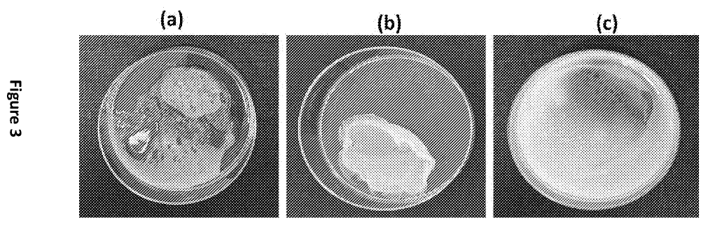

It is known in the state of the art that chitosan films are transparent, as showed in FIG. 2a. However, the incorporation of VCO into CHT solution and/or glycerol and/or AV promoted a significative reduction of the film transparency (FIG. 2b-d), and an increase in film thickness from 0.1 mm up to 0.5 mm. Therefore, the surface properties of the films, namely surface roughness, microstructure and hydrophilicity of the emulsion films can be modulated through VCO concentration.

In an embodiment, the emulsion films also have good stability and flexibility (FIGS. 2e and 2f), and can be used in tissue engineering applications.

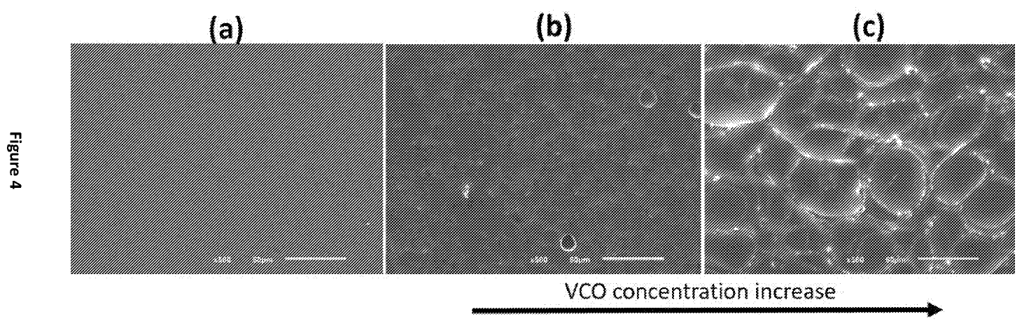

It was observed that emulsion films prepared by combining VCO with other polymers such as alginate, silk fibroin, and gelatin do not present good appearance (FIG. 3), which suggested a low interaction between the components of the emulsion. These findings also reinforce the critical role of chitosan as a natural surfactant into the chitosan/VCO based system.

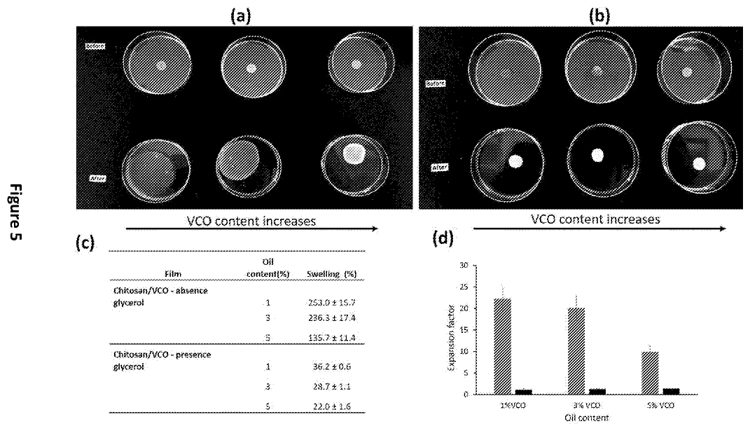

The surface morphology of the film samples was observed using a NanoSEM-FEI Nova 200 (FEG/SEM) scanning electron microscope. Before SEM (Scanning electron microscopy) analysis, samples were dried and further coated with gold using a Quorum/Polaron model E 6700 equipment, and the analysis was performed with an acceleration voltage of 5.00 kV and magnification from 150× to 4000×. An increasing surface roughness was observed at a different level on the surface of the films (FIG. 4b-c), when compared with the chitosan alone film (FIG. 4a).

Swelling studies on the emulsion films were performed, in water, to understand their water uptake ability (FIG. 5). It is possible to observe that after a short period of time immersed in the water (30 min), the emulsion films prepared without glycerol absorbed several times their weight, being characterized as superabsorbent materials (FIG. 5a). Due to the hydrophilic nature and the gel-forming ability of the polymer (chitosan), chitosan/VCO emulsion films of the present work are highly swellable. The swelling ability of the emulsion films may be modulated by the addition of the additives, like glycerol, as observed in Figure Sb.

In an embodiment, the swelling of the emulsion films was determined by calculating (W_{S}−W_{D})/W_{D}, where W_{S} represents the weight of the swelled sample and W_{D} the weight of the initial dry sample. Each experiment was repeated 3 times. Further, the films were dried at 60° C. for 48 h. The achieved results are illustrated in Figure Sc, and confirm the high swelling capacity of the emulsion films (up to 250%) in the absence of glycerol, and low swelling ability (up to 36%) in the presence of glycerol.

FIG. 5d is a graphical representation of the correlation between the initial oil content of the mixture and its expansion factor. The expansion factor refers to the degree of expansion in the linear dimension of an emulsion gel after being swollen in water to attain its equilibrium water content. In an embodiment, the dry films (0.6 mm diameter) were immersed in distilled water for 24 hours under gentle stirring in an orbital shaker at 50-60 rpm and room temperature. After 30 min of immersion, the samples were measured again, and the ratio between their initial size and their final size was considered for the expansion factor calculation.

U.S. Pat. No. 8,586,078 B2, describes emulsion gels and medical articles containing an omega-3 oil, wherein the achieved expansion factor was 1.25-1.5. It can be seen that the swelling capacity of the emulsion CHT/VCO films, expressed regarding the expansion factor, was much more pronounced for samples with low oil content (1-2%) and slightly decreases with an increase of the initial oil content (5% VCO). Moreover, the reduction of the expansion factor registered for the emulsion films that contain glycerol into its composition reinforces that the swelling ability of the films may be controlled by the addition of glycerol or its derivatives.

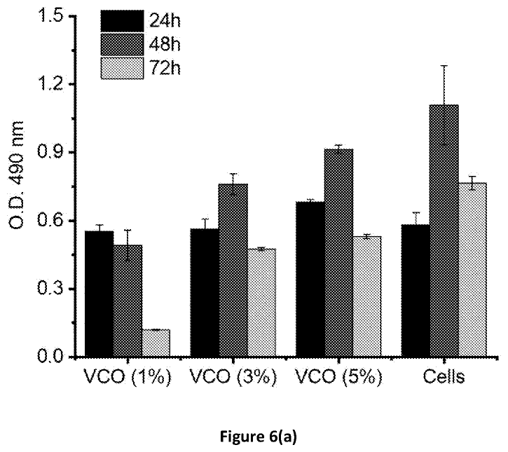

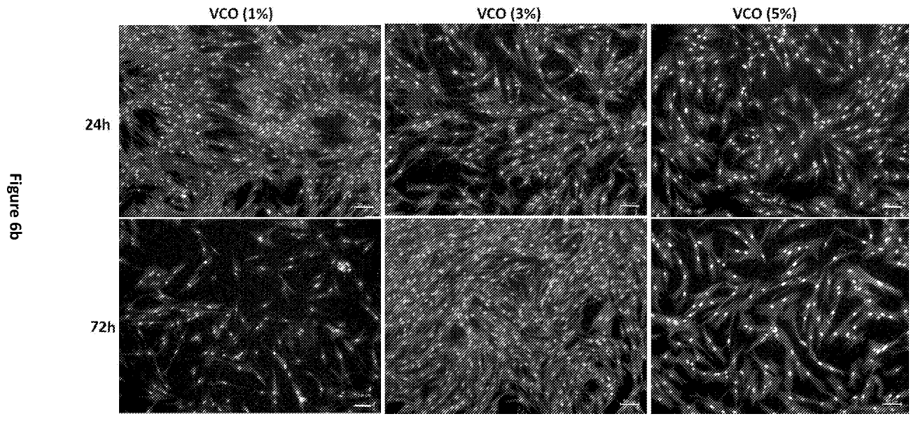

In an embodiment, cytotoxicity screening of the produced emulsion films was investigated following ISO 10993:2012. In FIG. 6, it is possible to observe cell viability assessed by MTS assay, and cell damage, assessed by F-actin staining, along 72 hours of culture. Cells were able to keep viable along the culture time with small differences between each extract tested and the control, thus proving their non-cytotoxicity behavior (FIG. 6a). Higher VCO concentration tended to result in higher cell viability as compared to other formulations, although a tendency to lower cell growth along the culture time. These observations were corroborated by F-actin staining, as illustrated in FIG. 6b.

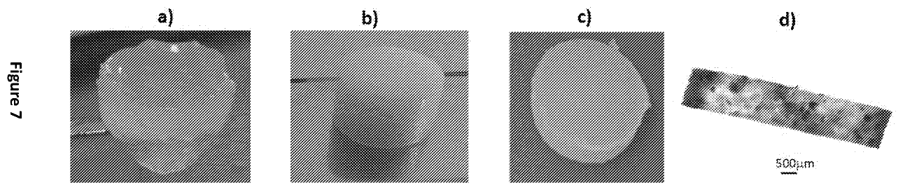

In an embodiment, in the course of developing the EC/VCO oleogels, it has been observed that there was no separation of the components of the emulsions during a shear process, such as spreading or mixing at temperatures below the gel temperature. The consistency of the EC/VCO oleogels may be varied from soft to rigid gels depending on molding temperature. FIG. 7 illustrates oleogels molded at 25° C. (FIG. 7a) and 4° C. (FIG. 7b). When compression molding is used, the resulting EC/VCO oleogels (FIG. 7c) presented good dimensional stability and a compact structure. Despite being compact structures, the application of salt leaching process combined with compression molding resulted in unique porous oleogels structures (FIG. 7d) with a pore size ranging between 100-500 μm, suggesting that those architectures would allow not only the successful seeding of cells but also their proliferation into the structure.

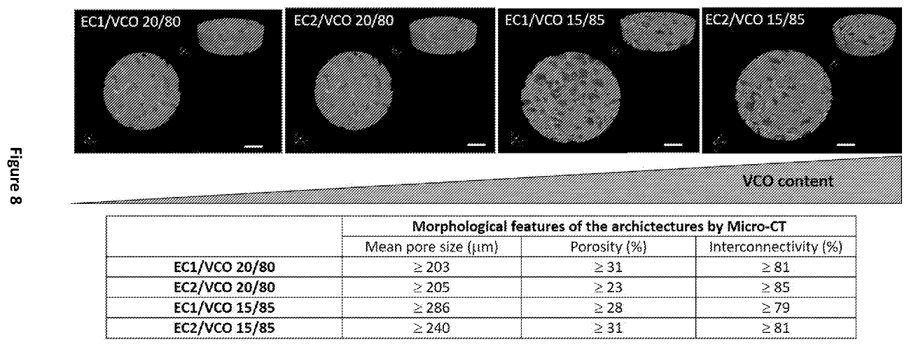

In an embodiment, micro-CT analysis was used to obtain quantitative information on the 3D architecture of the EC/VCO based porous oleogels (FIG. 8). It was demonstrated that oleogels presented moderated porosity and high interconnectivity. The differences in morphological features of the EC/VCO-based porous oleogels can be associated with different compositions and molding temperatures applied during their processing. Overall, the morphological features of the developed porous oleogels may help cells distribution and growth, and the transfer of nutrients.

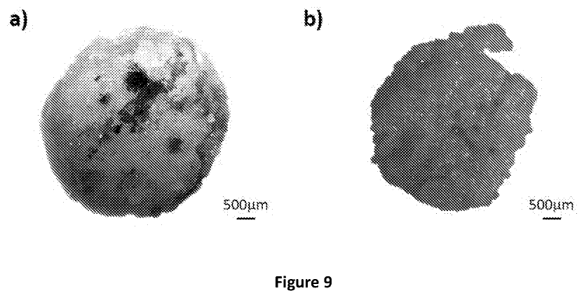

In an embodiment, oleogels (3D structures) were also prepared using EC/coconut oil (FIG. 9a) and EC/almond oil (FIG. 9b) and applying compression molding with salt leaching technique. In both structures, their morphological features were not well-defined. It seems that the interactions between components were not well established, in contrast to the evidenced in the EC/VCO-based porous oleogels.

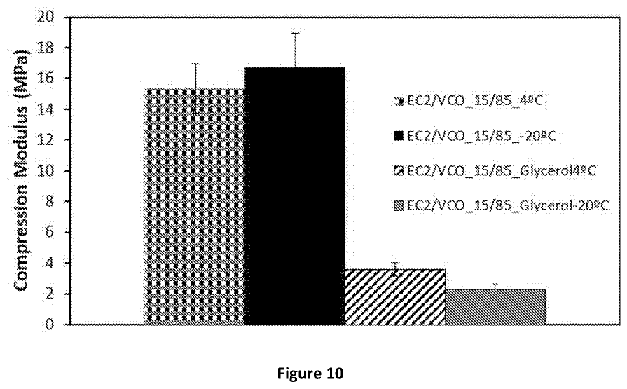

In an embodiment, the compression properties of the oleogels were measured using an Instron 5543 Universal Machine, (USA), and the tests were conducted using a 1 kN load cell and a crosshead speed of 1 mm·min⁻¹ (FIG. 10). Circular samples range of 1.87±0.06 mm of height and 8.01±0.02 mm of diameter were used and at least 6 samples per condition. The compression force was taken as the maximum stress in the stress-strain curve. In both tests the modulus was estimated from the initial slope of the stress-strain curve using the linear regression method. Samples were conditioned at room temperature for at least 48 h before testing. The average and standard deviations were determined using at least 6 specimens per condition.

In an embodiment, the compressive modulus properties of the oleogels obtained under compression mode are presented in FIG. 10. It is clear the existence of two distinct behaviors of EC/VCO oleogels being one composed by the ones without glycerol that presents higher stiffness and cooled at different temperatures, and the other group of the ones containing glycerol that showed lower stiffness. Moreover, slight differences were clearly observed when the cooling temperature was decreased. Differences observed in the mechanical properties of the oleogels can also be attributed to the packing of the fatty acids (VCO) within the EC gel network.

Thus, the results show the capability to tune the mechanical properties of the oleogels by using (a) different cooling temperatures of the system and (b) by using glycerol acting as a plasticizer agent to decrease the stiffness. The obtained mechanical properties are in the range of those materials that are applied for a wide range of tissue engineering applications.

In an embodiment, for the stem cells, isolation protocol, human adipose-derived stem cells (hASCs) were obtained from human adipose tissue after liposuction procedure, which was performed at Hospital da Prelada (Porto, Portugal), after patient's informed consent and under a collaboration protocol approved by the ethical committees of both institutions. To isolate the hASCs, the adipose tissue was submitted to the action of 0.05% collagenase type II (Sigma Aldrich, USA), under agitation for 1 hour at 37° C. Then, it was filtered with a strainer and centrifuged at 800 G for 10 min. After discarding the supernatant, pellets were resuspended in PBS and centrifuged at 350 G for 5 min. Finally, the cell pellet was resuspended in Minimum Essential Media a (α-MEM, Gibco, UK), supplemented with 10% fetal bovine serum (FBS, Invitrogen, USA), and 1% antibiotic/antimycotic (Invitrogen, USA). Cultures were maintained at 37° C. under a humidified atmosphere of 5% v/v CO₂ in air. The hASCs were selected by plastic adherence and passage at 80% confluence. In the different studies, hASCs in passage 4 were used.

In an embodiment, before cell culture testing, the samples (0.6 mm diameter) were sterilized in an ethylene oxide atmosphere. The evaluation of cytotoxicity of extracts of CHT/VCO films was performed as described in the ISO 10993-2012, using hASCs. First, hASCs were seeded in each well of a 96-well plate at a density of 10.000 cells per cm³. After 24 h of culturing, the CHT/VCO extracts were added to the top of cells. Additionally, a negative control (Ctrl-) was composed by extracts of latex and positive control (Ctrl+) composed of hASCs cells. Cultures were maintained at 37° C. under a humidified atmosphere of 5% v/v CO₂ in the air. Finally, at 24, 48, and 72 h of culture, cell growth and cell damage were analyzed as described below.

In an embodiment, in vitro tests of the EC/VCO oleogels were analyzed using mesenchymal stem cells derived from adipose tissue. For that purpose, cells were seeded on the surface of the materials at different cell density. The same number of cells was cultured in a 24-well plate and used as control. Cultures were maintained at 37° C. under a humidified atmosphere of 5% v/v CO₂ in air. At different time points, the cell's metabolic activity and cell damage were assessed.

In an embodiment, cell viability was assessed using the CellTiter 96® AQueous One Solution Cell Proliferation Assay (MTS, Promega, USA). At each time point, 24 h, 48 h, and 72 h cells were incubated with 20% v/v of MTS in culture medium without phenol red (Sigma Aldrich, USA) for 3 h at 37° C. The supernatant was then transferred to a new 96-well plate, and absorbance measurements were carried out using a microplate reader (Biotek Synergy HT) at 490 nm.

In an embodiment, cell damage was studied through F-actin staining. For that, cells were washed with phosphate buffer saline (PBS, Sigma Aldrich, USA), fixed with 10% Neutral Buffered Formalin (ThermoFisher Scientific, USA) for 15 min and permeabilized for 5 min with 0.1% v/v Triton X-100 (Sigma Aldrich, USA) in PBS. Afterward, samples were incubated for 30 min in 1% (w/V) of BSA (Sigma Aldrich, USA) in PBS to block unspecific binding. F-actin filaments were stained with Phalloidin-Tetramethylrhodamine B isothiocyanate (1:40, Sigma Aldrich, USA), and nuclei were counterstained with 1:5000 of the stock of 4,6-Diamidino-2-phenyindole, dilactate solution (DAPI, 1 mg/mL, Biotium, USA). Samples were analysed under an inverted fluorescence microscope (Zeiss Axio observer).

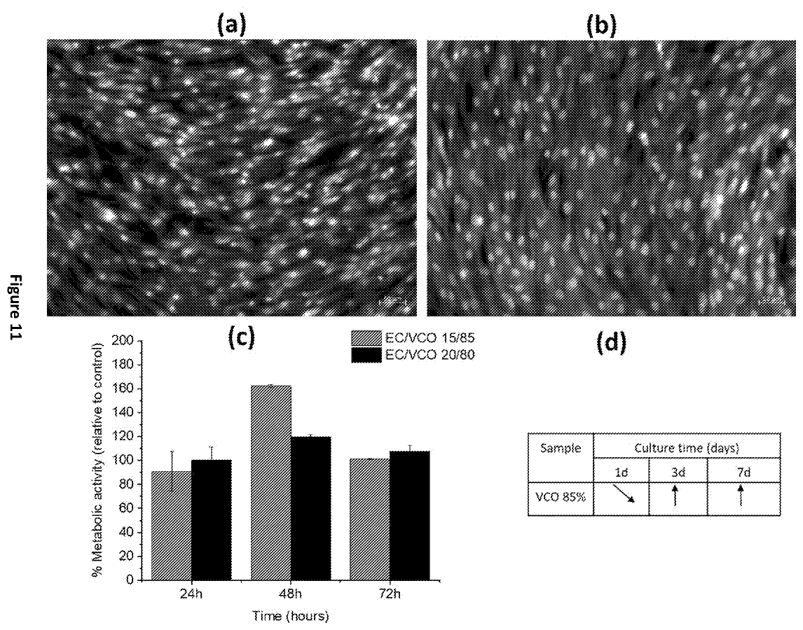

In an embodiment, the cytotoxicity of different EC/VCO-based porous oleogels formulations was investigated following ISO 10993:2012 and hASCs cells (FIG. 11a, b). Cells were able to proliferate along the culture time with small differences between each extract tested. FIG. 11c shows cell growth assessed by MTS assay. The results are presented in the percentage of cell growth in relation to negative control along the 72 hours of culturing. These observations were corroborated by F-actin staining, as illustrated in FIG. 11a-b.

In an embodiment, the biocompatibility of EC/VCO-based oleogels was also studied by direct contact with hASCs cells, used to provide a more physiologically relevant environment. In FIG. 11d it is depicted cell viability evaluated by metabolic activity determination using MTS assay along the time of culture (up to 7 days). It seems that the cell's metabolic activity was lower in day 1, but increasing from day 3 until day 7. These observations indicated that during the first 24 hours, the hASCs cells were adapting to the architectures, which make them more metabolic active.

The ability of hASCs to expand and differentiate into desired tissue types makes them an attractive cell source for tissue engineering applications. Moreover, these cells are easily obtained from adipose tissue, resulting from surgery. Therefore, the positive evaluation of the biological behavior of hASCs cells on both chitosan/VCO-based emulsion films and EC/VCO based oleogels, suggested that the developed matrices can serve as suitable platforms to cell culture and/or cell differentiation of hASCs cells in any specific tissue.

Chitosan/VCO emulsion films are characterized as superabsorbent materials. This feature can be modulated by the addition of glycerol or other derivatives to the emulsions;

Ethylcellulose/virgin coconut oil oleogels with different textures can be produced using gelation or compression molding process;

Compression molding combined with salt leaching process showed to be an effective method to produce ethylcellulose/virgin coconut oil porous oleogels;

The presence of additives as glycerol in the preparation of the formulations influenced: i) positively the interaction between the constituents of the emulsion system; ii) increases the flexibility of the films; iii) Increases the hydrophilicity of the oleogels, and iv) modulates the mechanical properties of the oleogels;

The stiffness of the EC/VCO oleogels can be tuned by (a) decreasing the cooling temperature from 37° C. up to −20° C., (b) the use of a plasticizer agent such as glycerol and (c) the addition of salt particles to create porosity into the oleogel structure;

The proposed compositions are useful for tissue engineering applications.

The biological assessment of the produced EC/VCO oleogels using hASCs suggested that the applicability of them could be addressed by hASCs cell differentiation.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim.

Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skilled in the art that a contradiction or inconsistency would arise.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

The disclosure should not be seen in any way restricted to the embodiments described, and a person with ordinary skill in the art will foresee many possibilities to modifications thereof.

The above-described embodiments are combinable.

The following claims further set out particular embodiments of the disclosure.

### REFERENCES

(1) Binsi P K, Ravishankar C N, Srinivasa Gopal T K. Development and characterization of an edible composite film based on chitosan and virgin coconut oil with improved moisture sorption properties. J Food Sci. 2013 April; 78(4):E526-34, 2013.
(2) Remington: The Science and Practice of Pharmacy, 19th Edition (1995), ISBN: 0-912734-04-3, Chapter 21, pages 282-291).

1. A composition for tissue engineering and/or regenerative medicine, comprising:
(i) a compound selected from the group consisting of chitosan, aloe vera, ethylcellulose, cellulose derivatives, and mixtures thereof, and,
(ii) coconut oil.

2. The composition of claim 1, comprising 1%-85% (v/v) of coconut oil.

3. The composition of claim 1, further comprising an additive, wherein the additive does not comprise surfactants.

4. The composition of claim 3, wherein the additive is selected from the group consisting of a salt, glycerol, glycerine, and mixtures thereof.

5. The composition of claim 3, comprising at least 1% (w/w) of the additive.

6. The composition of claim 1, in the form of a film, a membrane or an oleogel.

7. The composition of claim 6, comprising at least one of the following combinations:
chitosan and virgin coconut oil;
chitosan and coconut oil;
chitosan, aloe vera, and virgin coconut oil; and
chitosan, aloe vera, and coconut oil.

8. The composition of claim 7, comprising:
94-99% (v/v) of chitosan; 1-5% (v/v) of coconut oil;
and optionally 1% (v/v) of a plasticizer comprising glycerol.

9. The composition of claim 7, further comprising 31-33% of aloe vera.

10. The composition of claim 1, comprising chitosan and aloe vera, wherein the volume ratio of chitosan to aloe vera is 1.5:1-2.5:1.

11. The composition of claim 6, comprising:
ethylcellulose and virgin coconut oil;
or ethylcellulose and coconut oil.

12. The composition of claim 11, comprising:
15-30% (w/w) of ethylcellulose, cellulose derivatives, or mixtures thereof;
70-85% (w/w) of coconut oil; and a plasticizer comprising glycerol.

13. A method to prepare a film, membranes or oleogel comprising the steps of:
mixing two or more components of the following to form a mixture coconut oil, chitosan, aloe vera, ethylcellulose, cellulose derivatives, glycerol, glycerine, and a salt thereof, wherein at least one of the components is coconut oil;
homogenizing the mixture;
solvent casting or freeze-drying the homogenized mixture; and
optionally compression moulding or gelation.

14. The method of claim 13, further comprising the step of adding salt particles of suitable size in the mixture.

15. The method of claim 13, further comprising the step of salt leaching, and the step of immersion in distilled water for several hours or several days to remove the salt particles.

16. The method of claim 13, further comprising the step of drying at room temperature.

17. (canceled)

18. The method of claim 13, further comprising the step of freeze-drying, wherein the freeze-drying is at a temperature ranging between −20° C. and −196° C.

19. (canceled)

20. (canceled)

21. (canceled)

22. The composition of claim 2, comprising 1%-5% (v/v) coconut oil.

23. (canceled)

24. (canceled)

25. (canceled)

26. (canceled)

27. (canceled)

28. The composition of claim 1, wherein the coconut oil is virgin coconut oil.

29. A method of culturing cells, comprising adding the cells to the composition of claim 1.
